(19) United States
(12) Patent Application Publication
(10) Pub. No.: US 2017/0202711 A1
(43) Pub. Date: Jul. 20, 2017

(54) WOUND TREATMENT SYSTEM AND METHOD

(71) Applicants: Andrei CERNASOV, Ringwood, NJ (US); Nathalie CERNASOV, Ringwood, NJ (US); Andre CERNASOV, Ringwood, NJ (US)

(72) Inventors: Andrei CERNASOV, Ringwood, NJ (US); Nathalie CERNASOV, Ringwood, NJ (US); Andre CERNASOV, Ringwood, NJ (US)

(21) Appl. No.: 15/408,795

(22) Filed: Jan. 18, 2017

Related U.S. Application Data
(60) Provisional application No. 62/280,510, filed on Jan. 19, 2016.

(51) Int. Cl. A61F 13/00; A61B 5/145; A61B 5/01; A61B 5/00

(52) U.S. Cl. CPC A61F 13/00021; A61F 13/00008; A61F 13/00068; A61B 5/6802; A61B 5/4836; A61B 5/14539; A61B 5/01; A61F 13/00987; A61F 2013/00182

(57) ABSTRACT

A bandage for treating a wound includes a carrier plate and a peripheral support to affix to skin area surrounding a wound. The carrier plate and the peripheral support define an enclosure encompassing the wound and provide access to the wound for monitoring and treatment.

### CLAIM FOR PRIORITY

The present Application for Patent claims priority to U.S. Provisional Application No. 62/280,510 entitled “WOUND TREATMENT SYSTEM AND METHOD” filed Jan. 19, 2016, assigned to the assignee hereof and hereby expressly incorporated by reference herein.

### TECHNICAL FIELD

The disclosed apparatus and methods generally relate to wound treatment, particularly, bandages for treating chronic wounds.

### BACKGROUND

Bandages are known to include a piece of material used either to support a medical device such as a dressing or splint and/or restrict the movement of a part of the body. A dressing is a sterile pad or compress applied to a wound to promote healing and/or prevent further harm. A dressing is designed to be in direct contact with the wound, as distinguished from a bandage, which is most often used to hold a dressing in place. Other types of bandages are used without dressings, such as elastic bandages that are used to reduce swelling or provide support to a sprained ankle. Tight bandages can be used to slow blood flow to an extremity, such as when a leg or arm is bleeding heavily.

The estimated costs of treating chronic wounds range from 18 to 50 billion dollars per year. The average cost is over $3900 per wound while the average length of each treatment is 15 weeks. The average length of hospital stays for pressure ulcer treatments is 13 days.

A vast majority of treatments involve periodic cleanings, visual monitoring of wounds, and packing of wounds with a dressings, devices, and compounds, depending on the depth of the wound, presence of infection, presence of discharges, and state of the healing process. The timing of treatments varies, from daily to weekly, depending on the state of the wound.

### BRIEF DESCRIPTION OF DRAWINGS

The disclosed wound treatment device and method is described in detail in the following description with reference to the examples illustrated in the following figures.

FIG. 1 illustrates an area of skin protected with a wound treatment device, (hereinafter referred to as a ‘bandage”), according to an example of the present disclosure.

FIGS. 2a and 2b illustrate the bandage in FIG. 1 as applied to a wound area according to an example.

FIGS. 3a and 3b illustrate the bandage depicted in FIG. 1, including biocompatible material attached to a cover plate of the bandage according to an example.

FIGS. 4a and 4b illustrate the biocompatible material depicted in FIGS. 3a and 3b according to examples of the present disclosure.

FIGS. 5a and 5b illustrate the bandage including ports in the cover plate according to examples of the present disclosure.

FIGS. 6a and 6b illustrate the bandage, including test and treatment wells, according to an example of the present disclosure.

FIGS. 7a and 7b illustrate the bandage according to examples of the present disclosure.

FIGS. 8a and 8b illustrate the bandage as including electrical components and pad protrusions according to an example of the present disclosure.

FIG. 9 illustrates additional details of the bandage depicted in FIGS. 8a and 8b.

FIGS. 10a and 10b illustrate a mechanism to pump fluid through a wound enclosure of the bandage according to an example of the present disclosure.

FIGS. 11a and 11b illustrate the bandage of FIG. 1, including at least one wireless sensor, according to an example of the present disclosure.

FIGS. 12a and 12b illustrate the bandage of FIG. 1, according to other examples of the present disclosure.

FIGS. 13a, 13b, and 13c illustrate the bandage according to the present disclosure that facilitates visual monitoring of the wound.

FIGS. 14a and 14b illustrate another embodiment of the bandage of FIG. 1, according to an example of the present disclosure.

FIG. 15 illustrates a flowchart for designing, fabricating, and using the bandage of FIG. 1, according to an example of the present disclosure.

### DETAILED DESCRIPTION

For simplicity and illustrative purposes, the principles of the embodiments are described by referring mainly to examples thereof. In the following description, numerous specific details are set forth in order to provide a thorough understanding of the embodiments. It is apparent that the embodiments may be practiced without limitation to all the specific details. Furthermore, the embodiments may be used together in various combinations.

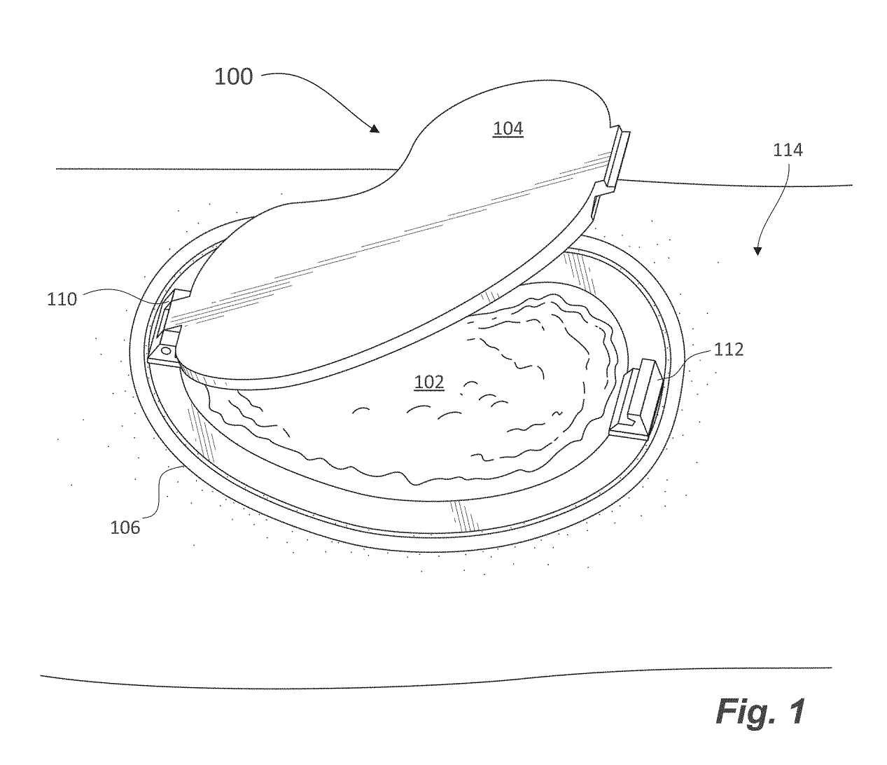

FIG. 1 depicts an example of a bandage 100 for treating chronic wounds. The bandage 100 is adaptable to the unique characteristics of each wound (size, depth, drainage, infections). Bandage 100 provides comfort to the patient while minimizing the frequency of major interventions and/or product replacements.

Bandage 100 includes a carrier plate 104 matched to a peripheral support 106. In the example shown in FIG. 1, a peripheral support 106 is attached to non-wounded skin 114, i.e., healthy skin, surrounding wound 102. As shown in the example of FIG. 1, the carrier plate 104 is mounted to the peripheral support 106 via a hinge 110 and a latch 112. In another example, the carrier plate 104 and peripheral support 106 are integrated into a single fabricated unit.

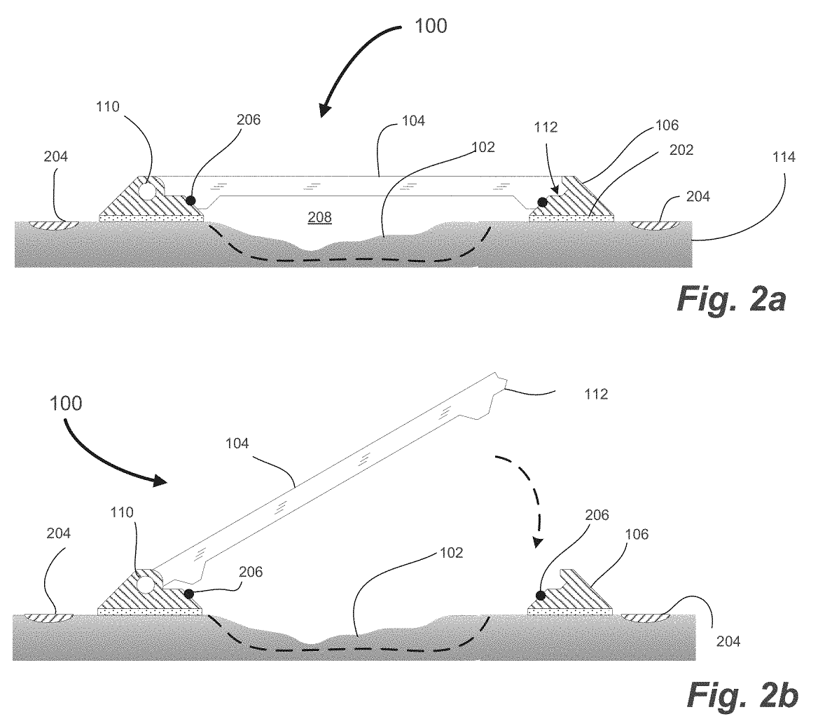

In FIGS. 2a and 2b, a bandage 100 is attached to the skin 114 using an adhesive 202 that affixes the peripheral support 106 to the surface of the skin 114. In another example, the carrier plate 104 may be pressed against the skin 114 by a strap (not shown) without a peripheral support. Markers 204 identify a perimeter of the wound 102. In an example, markers 204 are used to identify the wound area when the wound is scanned by optical devices including cameras and 3D scanning equipment. Additionally, the markers 204 may be used to accurately place a wound treating appliance, including bandage 100. At least a portion of carrier plate 104 is transparent and allows the care giver to observe the healing process through the transparent portion of the carrier plate 104. In an example, the care giver may access the wound 102 by opening latch 112 and rotating the carrier plate 104 around hinge 110 to expose the wound 102. A sealing gasket 206 runs along a contact edge between the carrier plate 104 and the peripheral support 106. The carrier plate 104 behaves as a treatment door that provides easy access to the wound 102 without requiring the unwrapping and wrapping of bandages or removal and reapplication of adhesive strips.

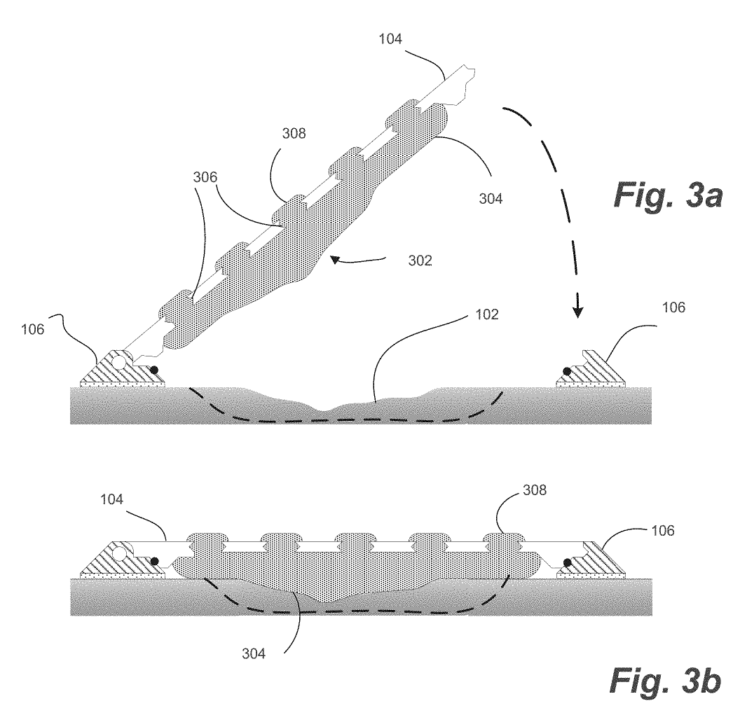

FIGS. 3a and 3b depict the carrier plate 104 including ports 306, which can be used to fasten biocompatible material 302, such as sponge or gauze used to absorb exudates, extending to the wound 102 to the carrier plate 104. The material 302 may be pre-shaped to present protrusions 308 which mate with the ports 306.

The wound enclosure 208, defined by the skin surface and inside surface of the carrier plate 104, is modeled and the biocompatible material 302 is shaped along cut line 304 to match the shape of the wound. In an example, the biocompatible material 302 is shaped to fill the wound enclosure 208, but not to apply pressure to the wound 102. If there is bleeding to be stopped, the biocompatible material 302 is shaped to apply pressure to a surface of the wound 102.

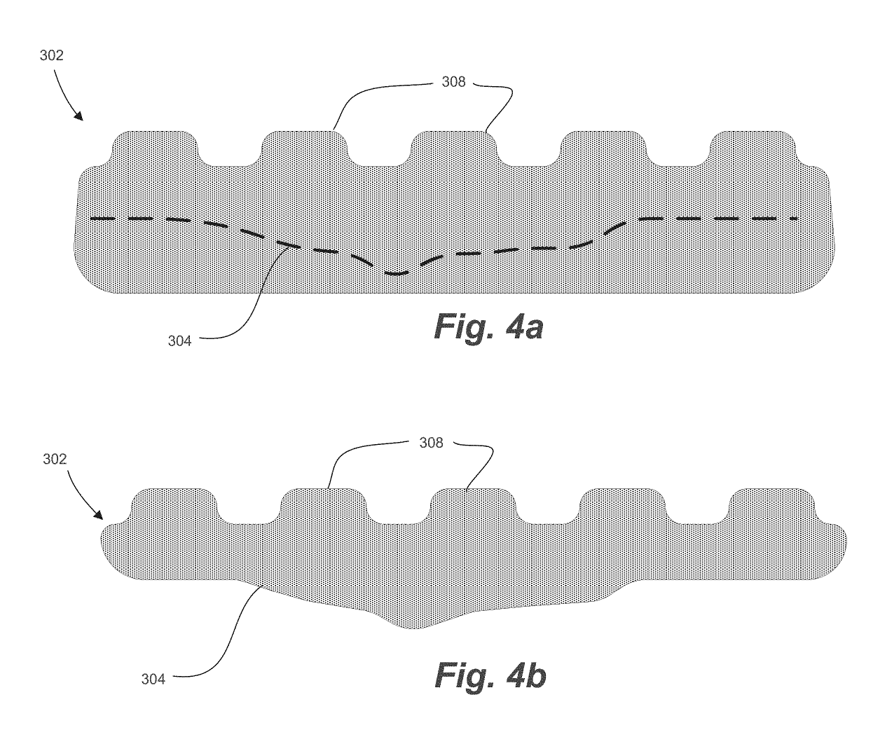

In an example, the biocompatible material 302 is fastened to a moveable portion of the carrier plate 104, which is snapped in its closed position as shown in FIG. 3b. FIGS. 4a and 4b illustrate biocompatible material 302 prior to, and after being shaped along cut line 304.

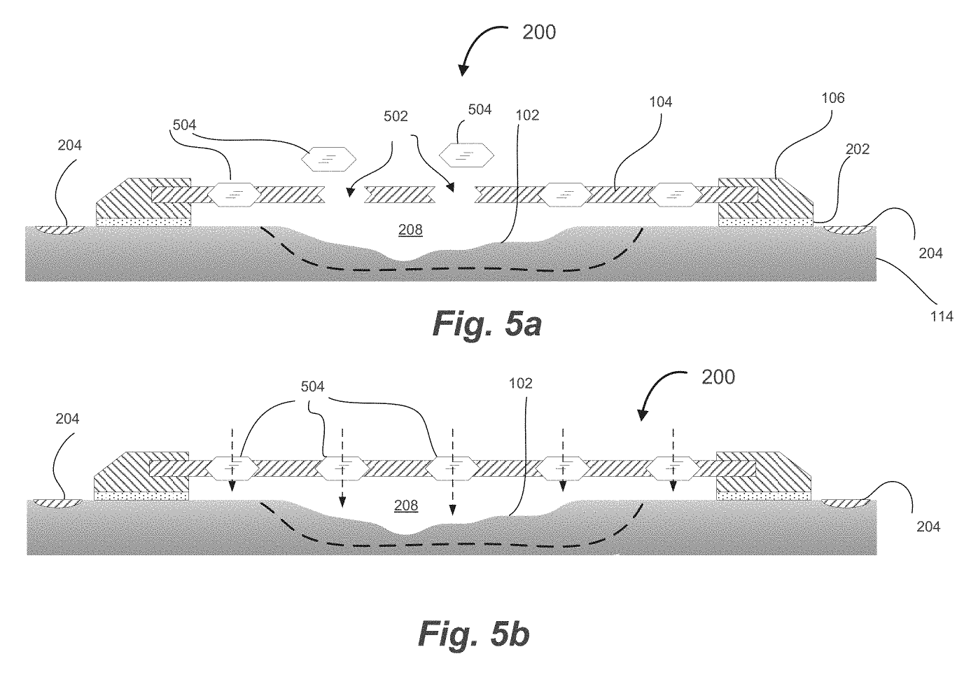

In an example, FIGS. 5a and 5b depict bandage 200, in which carrier plate 104 and peripheral support 106 are fabricated as a single unit. Peripheral support 106 is attached to the skin 114 surrounding the wound 102 using an adhesive 202 or alternate mechanism, such as a strap (not shown) that secures the bandage 200 to the skin.

FIGS. 5a and 5b depict carrier plate 104 having multiple ports 502 that facilitate sensing and/or treatment of the underlying wound 102. In an example one or more of plugs 504 is transparent and is inserted into selected ports 502, depending on treatment requirements. In FIG. 5b, plugs 504 are transparent and allow for visual monitoring of the wound enclosure 208.

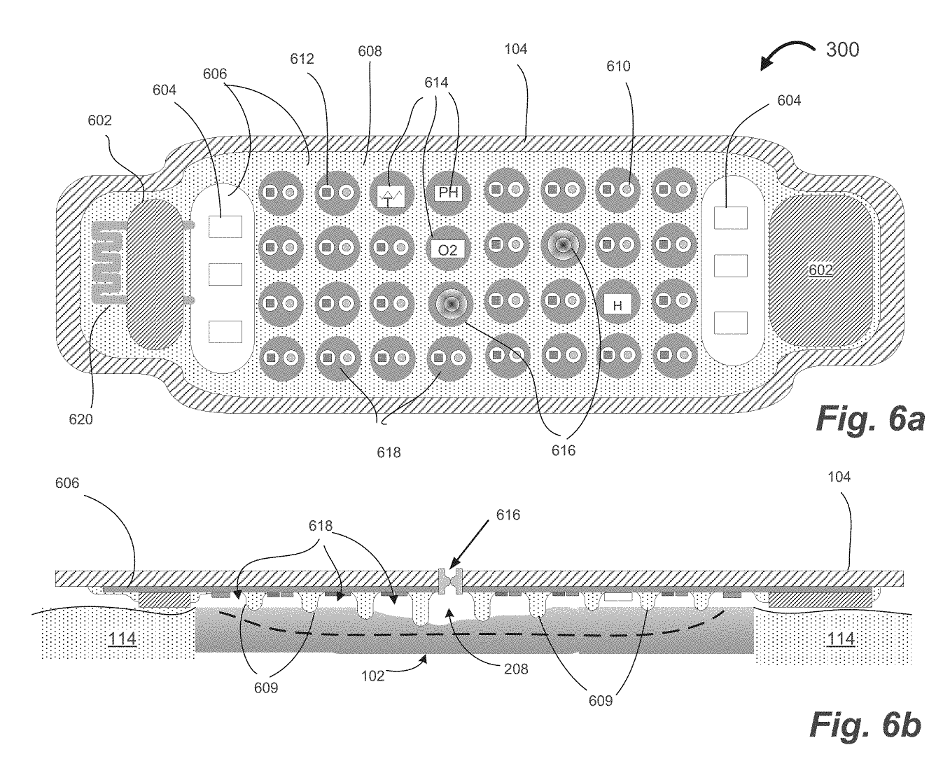

FIGS. 6a and 6b depict a bandage 300 that includes a closed loop monitoring and treatment system that includes sensing and actuating components 614 and intervention ports, i.e., test and treatment wells (hereinafter “wells”) 618 for treating chronic wounds. The sensing and actuating components 614 monitor and communicate to a care giver the status of the wound 102 on a continuous basis and wells 618 give the care giver access to the wound 102 on short notice and with minimal effort. The sensing and actuating components 614 may be interchanged and are appropriate to the type of wound being treated (pressure ulcer, burn, etc.).

In an example, wells 618 are defined by protrusions 609 extending from a pad 608 formed of biocompatible material 302 (see FIGS. 3-4) that is affixed to carrier plate 104. In the example shown, pad 608 and protrusions 609 are made of gauze, foam, or any other suitable material. In different examples, carrier plate 104 includes multiple wells 618. Although wells 618 are depicted as being cylindrical in shape, in other examples, wells 618 may take other shapes appropriate to the wound 102 and the specific treatment for the wound 102. Within each well 618, the sensing and actuating components 614 provide interaction with the wound 102.

Sensing and actuating components 614 may include an oxygen sensor (O2), a Ph sensor (PH), a temperature sensor (T), or other type of sensor, which may be positioned in the bandage 300 to provide information regarding the state of healing of the wound 102. Humidity sensor (H) may provide an indication of whether the wound enclosure 208, i.e., the area encompassed by the bandage 300, is properly insulated. Sensing and actuating components 614 are not limited to the sensors and actuators listed above and may be replaced or augmented by other sensors and actuators as needed to monitor and treat a wound. In other examples, laser diodes provide phototherapy effects. Pairs of LEDs 610 and photo detectors 612 may be incorporated to ascertain the color of the wound in the visible, as well as the infra-red spectrum.

The sensing and actuating components 614, as well as electronic components 604 that communicate with the sensing and actuating components 614, are, in an example, mounted on a circuit board 606, which is affixed to carrier plate 104. In examples, the circuit board 606 is flexible and the carrier plate 104 is fabricated as a single structure.

In an example, bandage 300 includes at least one input/output (I/O) channel 616 that extend through the carrier plate 104 and provide access to the wound 102. In an example, I/O channel 616 includes deformable sidewalls to provide a self-sealable access port to the wound 102 in order to extract tissue samples and/or apply treatments to the wound 102.

Still further, FIGS. 6a and 6b depict adhesive strips 602 to affix the bandage 300 to the skin 114. Further still, electronic components 604 include devices to facilitate communication with external devices via a radio frequency (RF) link that supports at least one of Wi-Fi, BLUETOOTH™, Near Field Communication (NFC) and other protocols. Antenna 620 may support these or similar communication channels.

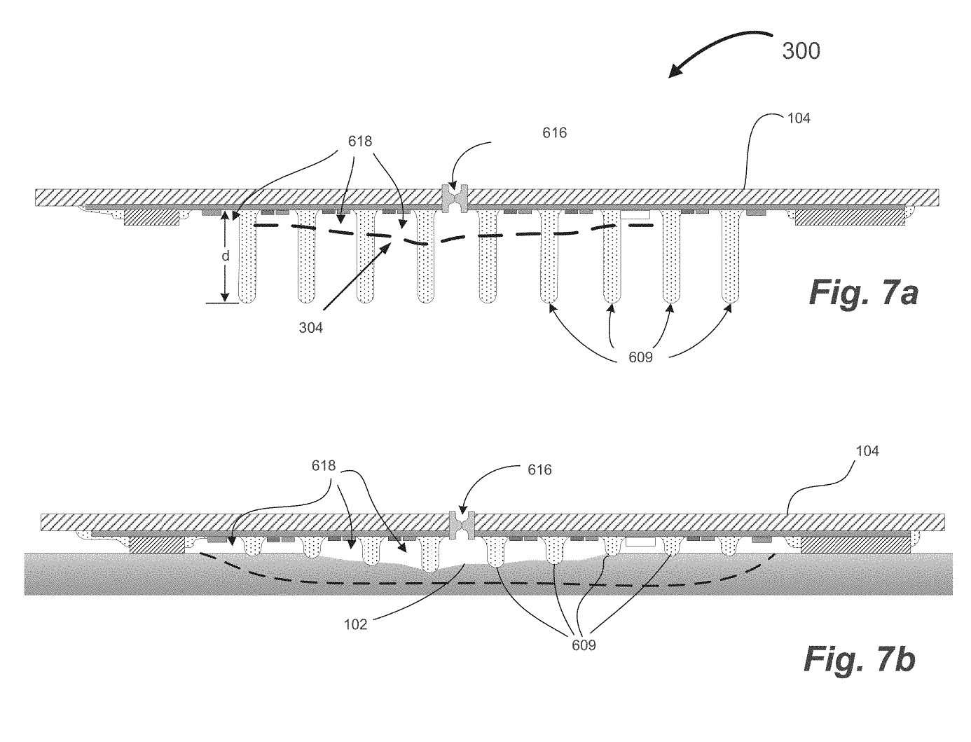

A wound 102 has its own shape and size. FIGS. 7a and 7b depict examples of a bandage 300 in which a depth (d) of each well 618 may be individually adjusted for a specific wound 102 being treated. FIG. 7a shows the bandage 300 having the protrusions 609 in an original and unadjusted state. FIG. 7b depicts the protrusions 609 cut based on a 3D model of the wound, so that the wells 618 conform to the wound 102.

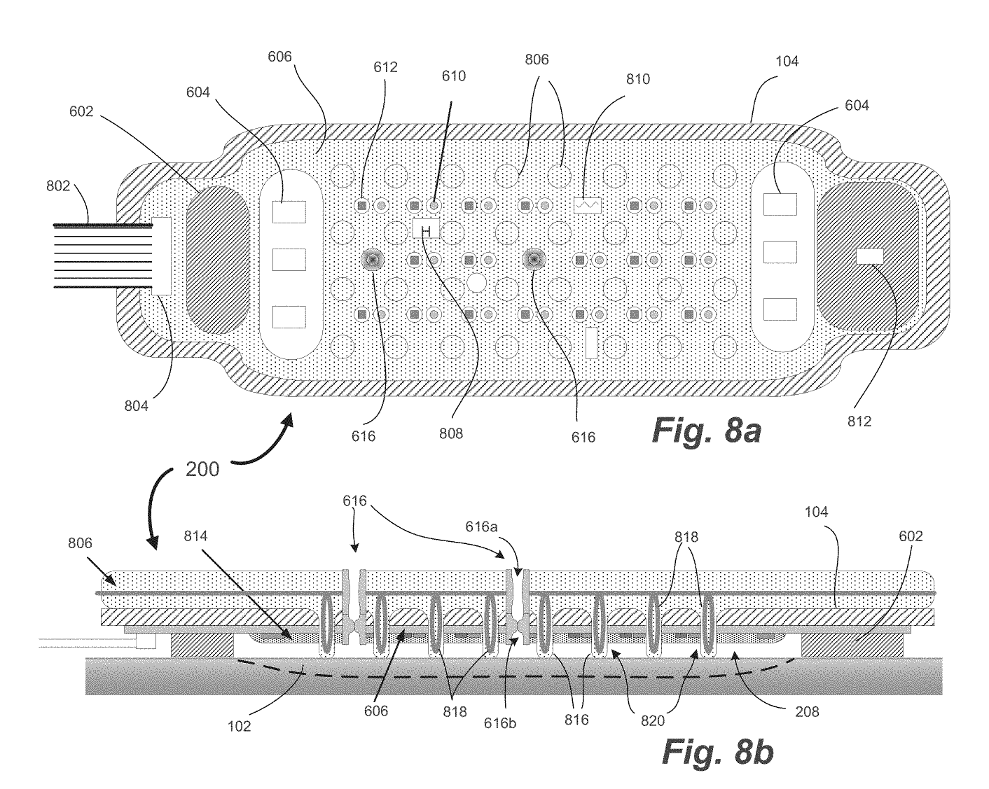

FIG. 8a depicts an example of bandage 200 that includes a cable 802 attached to a connector 804 in electrical communication with electronic components 604 and sensors and actuators disposed on the carrier plate 104. The sensors and actuators include, but are not limited to photo detector 612, light emitting diode (LED) 610, humidity sensor 808, thermometer 810, and a Galvanic Skin Response Sensor (GSR) 812 to measure electrical conductance of the skin.

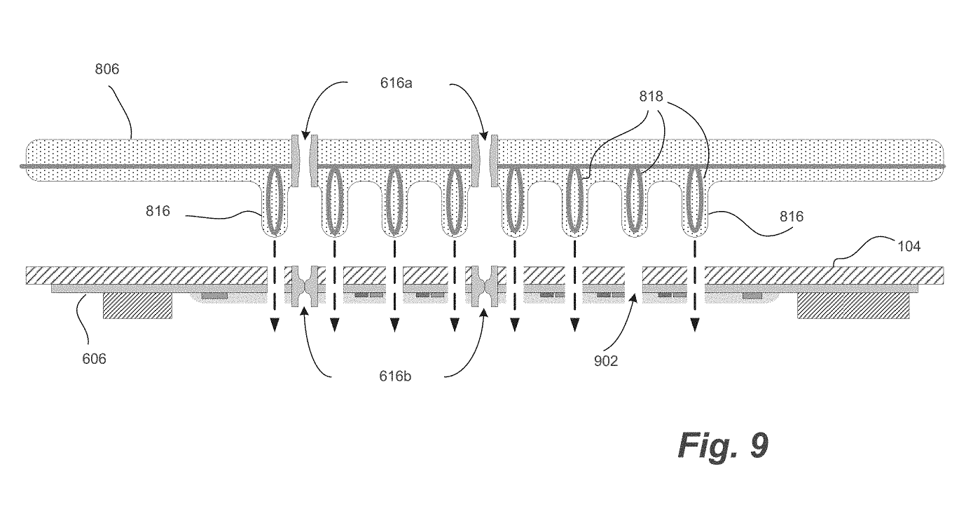

FIG. 8b further depicts biocompatible pad 806 making contact with the wound via pad protrusions 816. In an example biocompatible pad 806 is secured to the carrier plate 104 via pad protrusions 816, which may be forced through apertures 902 (FIG. 9) within carrier plate 104 and/or circuit board 606. The pad protrusions 816 include a spring 818 or similar feature to hold the biocompatible pad 806 in place applying tension against an inner surface of the apertures 902. I/O channels 616 are formed by the alignment of I/O channels 616a and I/O channel 616b, through biocompatible pad 806, carrier plate 104, and circuit board 606. In an example, a biocompatible coating 814 isolates the electronic components 604 mounted on the circuit board 606 from the wound enclosure 208 and to avoid contact with wound 102.

FIG. 9 depicts an example of the bandage 200 shown in FIGS. 8a and 8b in which biocompatible pad 806 is separated from, and is reattached to the carrier plate 104 as required. The removed biocompatible pad 806 may be discarded, recycled, cleaned, and/or reused. As depicted in FIG. 9, spring loaded pad protrusions 816 are urged through apertures 902 in the carrier plate 104 and circuit board 606.

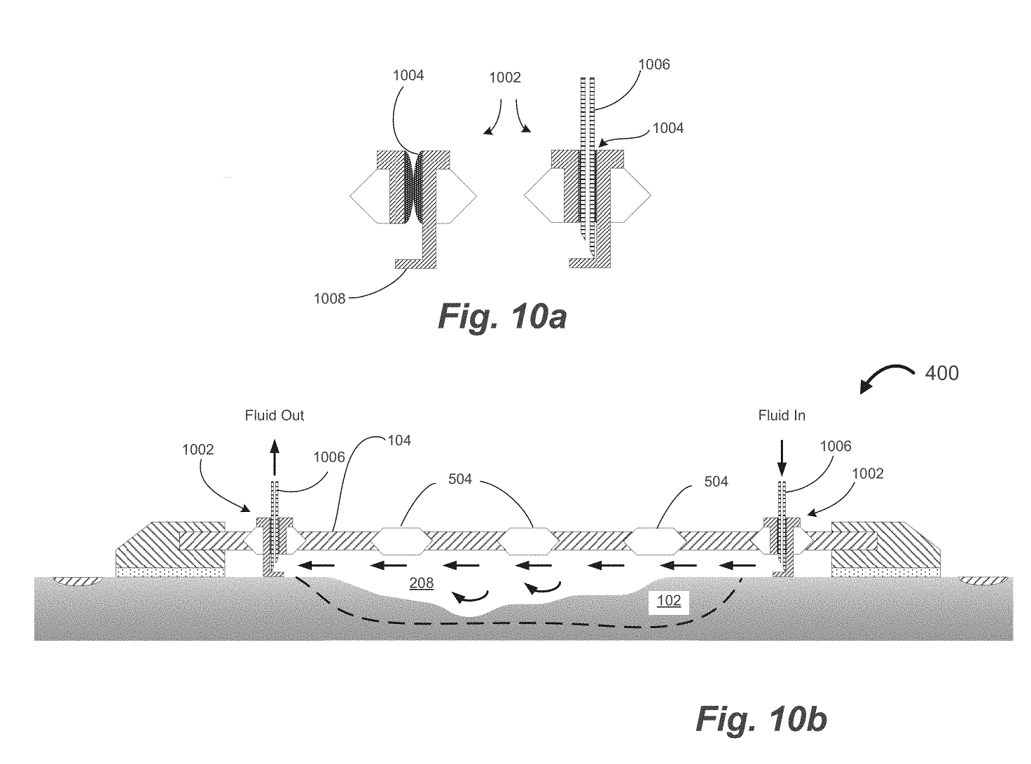

FIG. 10a depicts an example of a bandage 400 similar to the bandage 200 in FIGS. 5a and 5b. Bandage 400 includes plugs 504 and needle ports 1002. Needle ports 1002 allows a needle 1006 to access to the wound enclosure 208. Needle ports 1002 features a seal 1004, which is flexible and compressible to allow needle 1006 passage through the seal 1004 when the needle 1006 is pressed through the seal 1004. Needle port 1002 has a hard needle stop 1008 to stop the needle 1006 from accidentally sticking the skin underneath the needle port 1002. In the example shown in FIG. 10b, two needle ports 1002 circulate a fluid over the wound 102. In an example, the fluid may be a cleansing solution, an antibiotic gel, or other fluid treatment component. Any remaining I/O ports may be sealed by plugs 504.

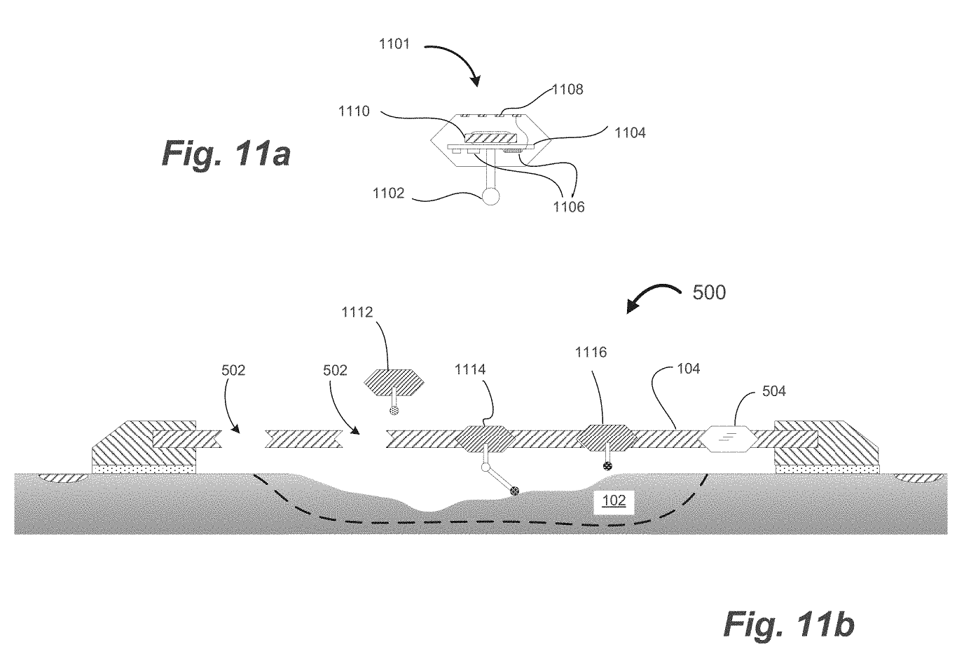

FIG. 11a depicts a bandage 500 that includes a wireless sensor 1101 designed to fit within the ports 502 formed within carrier plate 104. Wireless sensor 1101 includes a sensing device 1102, electronic circuitry 1106 mounted on a printed circuit board 1104, a battery 1110 and an antenna 1108. Wireless sensor 1101 communicates readings of sensing device 1102 to an external device (not shown). Examples of the wireless sensor 1101 are depicted in FIG. 11b and include wireless temperature sensor 1112, wireless Ph sensor 1114, and wireless oxygen sensor 1116. Plug 504 is transparent and allows viewing of the wound 102 without requiring the removal of the bandage 500.

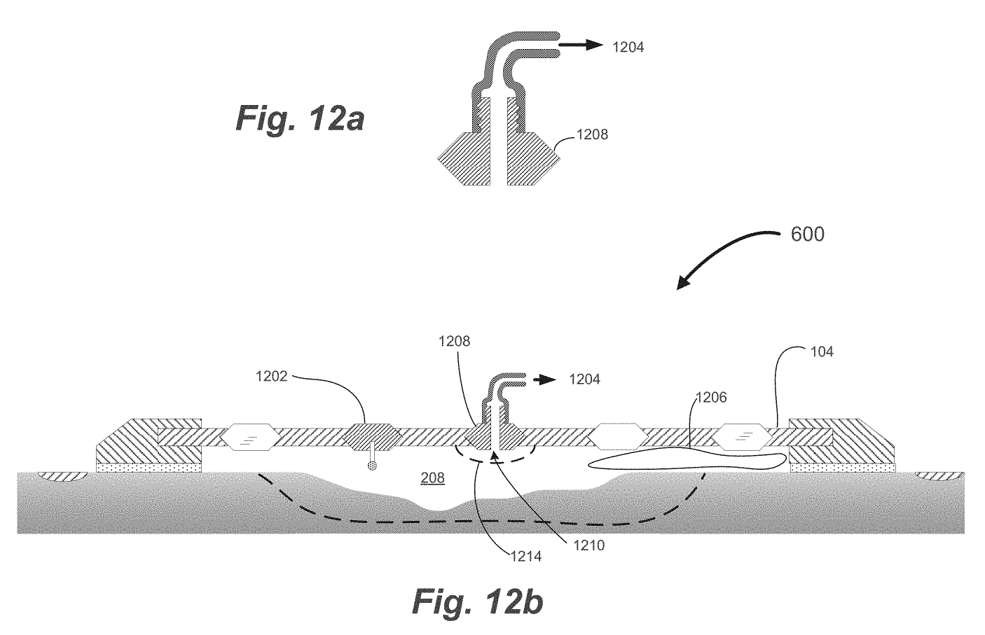

FIGS. 12a and 12b depict an example of a bandage 600 to deliver Negative Pressure Wound Therapy (NPWT) to the wound 102. As shown in FIGS. 12a and 12b, plug 1208 includes a pump inlet 1210 that couples the wound enclosure 208 to an external vacuum pump 1204. In an example, pressure sensor plug 1202 monitors the process and a filter 1214 protects the pump inlet 1210 to the plug 1208 from being clogged by packing material 1206.

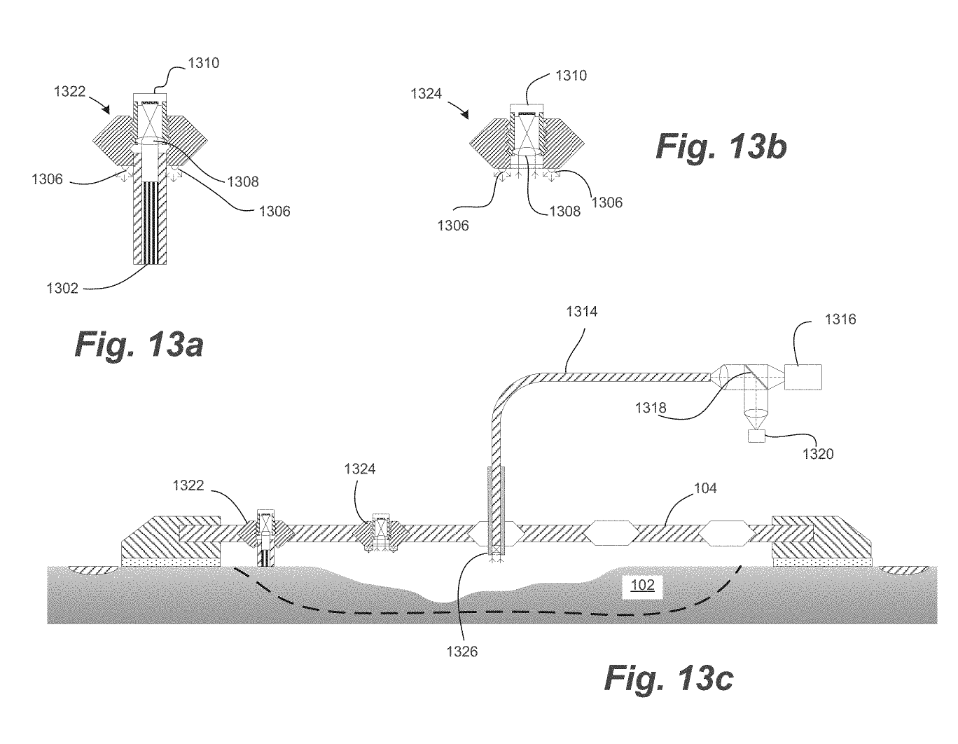

FIGS. 13a-c depict at least one optical device to allow visual monitoring of the wound. FIGS. 13a depicts a contact camera 1322 that includes an optical fiber bundle 1302 that is brought in contact with the wound 102. Illumination of the wound 102 is provided by at least one LED 1306. An optical lens 1308 allows a surface image of the wound 102 to be transmitted through the optical fiber bundle 1302 to optical sensor 1310. FIG. 13b depicts a camera plug 1324 that in an example includes LED(s) 1306 and lens 1308.

FIG. 13c depicts a remote optical processor arrangement in which an image of the wound 102 is captured by lens 1326 and is transmitted through a coherent optical fiber bundle 1314 to a remote optical sensor 1316. In examples, the remote optical sensor 1316 includes a camera, a spectrometer, a near infrared imager, and other sensing devices. In an example, FIG. 13c depicts the wound 102 illuminated by a source 1320 through beam splitter 1318.

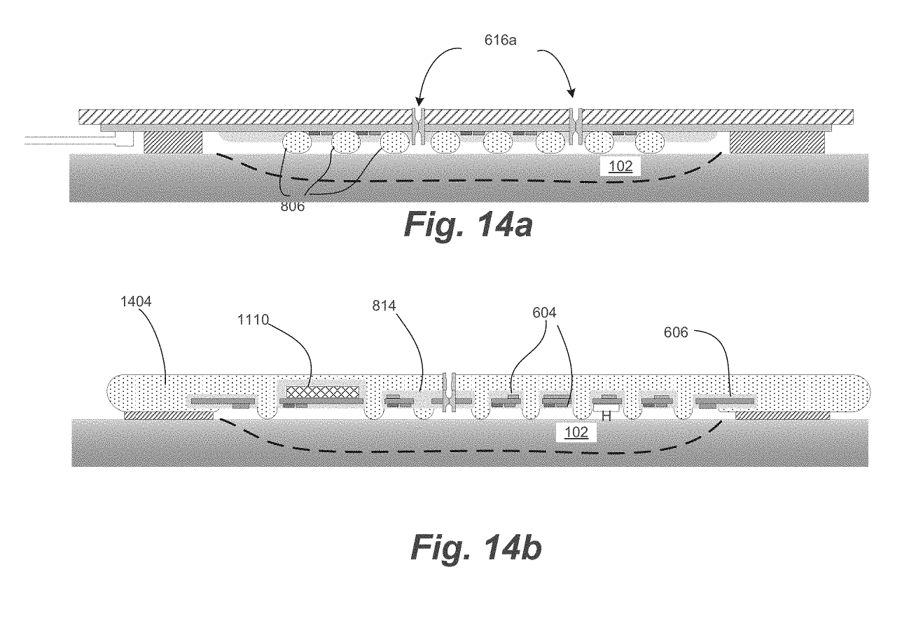

FIG. 14a depicts an example in which individual biocompatible pads 806 are placed permanently or temporarily, onto the carrier plate 104 and/or circuit board 606. In FIG. 14b, the circuit board 606 is double sided and has electronic components 604, e.g., sensors, memory devices, and a processor, mounted on both sides of the circuit board 606. In an example, the circuit board 606 and the electronic components 604 mounted thereon are enclosed in a pouch 1404 made of biocompatible material.

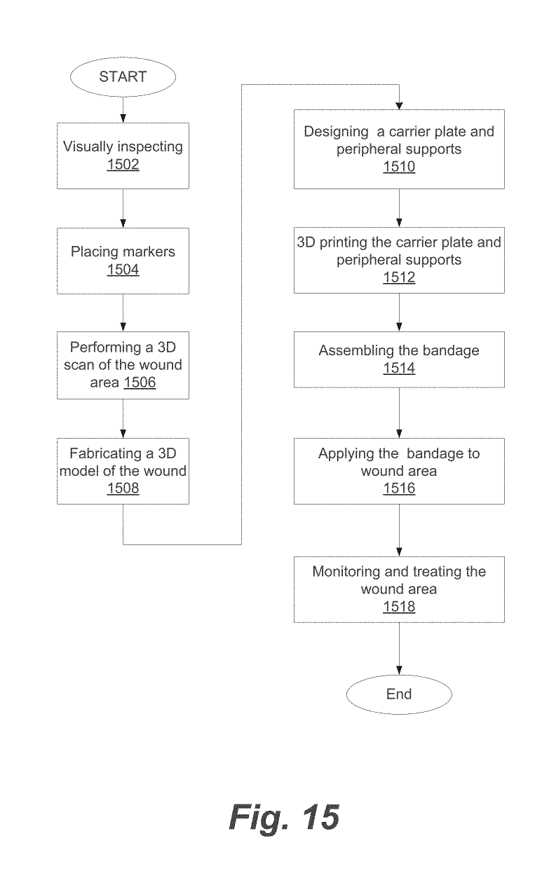

FIG. 15 depicts an example of a flowchart depicting a method of constructing and applying a bandage 100 that is custom made for a specific wound 102 on the surface of the skin 114. At step 1502, the method includes identifying a perimeter of the wound 102.

At step 1504, markers 204 are placed on the skin at various areas of interest, including skin areas which are determined healthy enough to support the peripheral support 106. In embodiments, skin areas, e.g., which cannot be touched, are differently marked, such as with a different color.

At step 1506, a 3-dimensional (3D) scan of the wound is performed.

At step 1508, a 3D model of the wound 102 is created based on the markers 204 and on the results of the 3D scan. A visual comparison of the wound 102 and the 3D model of the wound 102 may be performed to verify that the 3D model is a correct representation of the wound 102.

At step 1510, the 3D model of the wound 102 is communicated to an engineering or bio-engineering professional in order to design the carrier plate 104 and the peripheral support 106.

At step 1512, the carrier plate 104 and the peripheral support 106 are fabricated. The fabrication is done by various fabrication techniques including 3D printing.

Under certain circumstances, e.g., when the wound 102 is small, the scanning of the wound (step 1506), the building of the 3D model (step 1508), and the fabrication of the carrier plate and the peripheral support may not be necessary. Under these circumstances, pre-fabricated versions of the carrier plate 104 and the peripheral support 106 may be quickly assembled to treat the wound.

At step 1514, assembling the bandage is described as attaching one or more of biocompatible material 302, electronic components 604, sensors and actuators 614, antenna 620, and circuit board 606, to the carrier plate 104.

At step 1516, the peripheral support 106, the carrier plate 104, and bandage 100 is applied to the wound area, and as shown in FIGS. 6b, 7a, and 7b, applying bandage 300 to the wound may include adjusting a height d of biocompatible pad 608 to the contours of wound 102. In an embodiment, the peripheral support 106 is secured to the skin 114 via adhesive 202.

At step 1518, monitoring and treating the wound area are facilitated by the capabilities of the bandage 100, which in some embodiments, does not require opening the plate and exposing the wound to the atmosphere.

In embodiments, electronic components 604 include one or more hardware processors, a computer readable medium, which may be non-transitory, such as hardware storage devices (e.g., RAM (random access memory), ROM (read only memory), EPROM (erasable, programmable ROM), EEPROM (electrically erasable, programmable ROM), and flash memory). The methods, functions and other processes described herein may be embodied as machine readable instructions stored on the computer readable medium.

While the embodiments have been described with reference to examples, various modifications to the described embodiments may be made without departing from the scope of the claimed embodiments.

1. A bandage to be applied to a wound on a surface of a body, comprising:
a peripheral support to affix to skin area surrounding the wound; and
a carrier plate mounted to the peripheral support, wherein the carrier plate and peripheral support define an enclosure encompassing the wound and provide access to the wound.

2. The bandage according to claim 1, comprising a hinge and a latch to secure the carrier plate to the peripheral support, wherein the hinge is to allow the carrier plate to be opened to allow access to the wound.

3. The bandage according to claim 1, wherein the bandage includes:
a biocompatible material disposed within the defined enclosure, wherein the biocompatible material is attached to the carrier plate, and wherein the biocompatible material is pre-shaped to conform to a shape of the wound.

4. The bandage according to claim 1, the carrier plate including:
at least one port to allow access to the wound; and
a plug removeably inserted in the at least one port.

5. The bandage according to claim 4 wherein the plug comprises at least one of a transparent plug, a sensor, and an actuator.

6. The bandage according to claim 4, wherein the plug comprises at least one of a needle port, a temperature sensor, a Ph sensor, an oxygen sensor, a pump inlet, and an optical device.

7. The bandage according to claim 4, wherein the plug comprises a wireless sensor, the wireless sensor comprising at least one of a temperature sensor, a Ph sensor, and an oxygen sensor.

8. The bandage according to claim 7, wherein the plug comprising the optical lens further comprises a optical fiber bundle extending from the optical lens towards the wound.

9. The bandage according to claim 1, further comprising:
a biocompatible pad affixed to the carrier plate;
at least one of a test and treatment well defined by protrusions in the biocompatible pad; and
at least one of a sensor and an actuator affixed to the carrier plate.

10. The bandage according to claim 1, wherein the carrier plate includes a self-sealable access port to provide access to the wound.

11. The bandage according to claim 1, wherein the carrier plate includes a biocompatible pad, the biocompatible pad including pad protrusions urged through apertures defined within the carrier plate.

12. The bandage according to claim 1, wherein the carrier plate comprises at least one of a transparent window, a sensor and an actuator.

13. The bandage according to claim 1, wherein the peripheral support is integral to the carrier plate.

14. A method of treating a wound, comprising:
forming a bandage comprising a peripheral support to attach to skin surrounding the wound, and a carrier plate affixed to the peripheral support, the peripheral support and the carrier plate defining an enclosure encompassing the wound; and
at least one of monitoring and treating the wound via at least one port disposed within the carrier plate.

15. The method of claim 14, wherein forming the bandage comprises:
inserting a plug within the at least one port, the plug comprising at least one of a sensor plug, an actuator plug, and a transparent plug;
monitoring a status of the wound via the transparent plug and the sensor plug; and
treating the wound via at least one of the actuator plug and the at least one port.

16. The method of claim 15, wherein inserting the plug within the at least one port includes inserting at least one of an optical sensor, a pressure sensor, an oxygen sensor, a Ph sensor, a humidity sensor, a temperature sensor, pump plug, and a needle port within the at least one port.

17. The method of claim 14, wherein the carrier plate includes a biocompatible pad and a circuit board comprising electronic components, the biocompatible pad including pad protrusions urged through apertures defined within the carrier plate and the circuit board.

18. The method of claim 14, wherein forming the bandage comprises:
performing a 3-dimensional scan of the wound;
fabricating a 3-dimensional model of the wound based on the 3-dimensional scan of the wound; and
forming the bandage for the wound based on the 3-dimensional model.

19. The method of claim 14, wherein the carrier plate is hinged to the peripheral support.
